(19) United States
(12) Patent Application Publication
(10) Pub. No.: US 2024/0285931 A1
(43) Pub. Date: Aug. 29, 2024

(54) CONTROL FOR EXTRACORPOREAL CIRCULATORY SUPPORT

(71) Applicants: Xenios AG, Heilbronn (DE); Hochschule Offensburg, Offenburg (DE)

(72) Inventors: Matthias Heinke, Rudolstadt (DE); Johannes Hörth, Offenburg (DE); Steffen Eschinger, Frankfurt am Main (DE); Ivo Simundic, Wendlingen (DE); Caroline Bracker, Hanau (DE)

(21) Appl. No.: 18/007,418

(22) PCT Filed: Aug. 3, 2021
(86) PCT No.: PCT/EP2021/071702

§ 371 Date: Mar. 5, 2024

(30) Foreign Application Priority Data
Aug. 3, 2020 (DE) 10 2020 004 697.5

(51) Int. Cl. A61M 60/515; A61M 60/117; A61M 60/279; G16H 40/63

(52) U.S. Cl. CPC A61M 60/515; A61M 60/117; A61M 60/279; G16H 40/63; A61M 2230/04

(57) ABSTRACT

The present invention relates to control units for extracorporeal circulatory support as well as systems comprising such a control unit and corresponding methods. Accordingly, a control unit (10) for an extracorporeal circulatory support is suggested, which is configured to receive a measurement of an ECG signal (12) of a supported patient over a predetermined period of time and to provide said measurement for the extracorporeal circulatory support, wherein the ECG signal (12) comprises a signal level from at least one ECG lead (14A, 14B) for each time point within a cardiac cycle. The control unit (10) comprises an evaluation unit (16) which is configured to determine a signal difference (18) of a signal level of a current time point (12A) and a signal level of the preceding time point (12B) and to compare the signal difference (18) with a predetermined threshold value (20). The control unit (10) is further configured to provide the ECG signal (22) with a predetermined signal level (30) for the current time point and a predetermined number of subsequent time points (28), when the threshold value (20) is exceeded.

### TECHNICAL FIELD

The present invention relates to control units for an extracorporeal circulatory support as well as systems comprising such a control unit and corresponding methods.

### TECHNOLOGICAL BACKGROUND

If the pumping capacity or pumping function of the heart fails, a cardiogenic shock may occur, which, due to a reduction in cardiac output or cardiac ejection, may result in a reduced perfusion or blood flow to the end organs such as the brain, kidneys, and the vascular system in general. This acute heart failure results in an acute blood deficiency in the tissues and organs and thus an oxygen deficiency, also called hypoxia, which can lead to damage to the end organs. In most cases, such cardiogenic shock occurs as a result of a complication of acute myocardial infarction (AMI) or cardiac infarction. However, such life-threatening situations can also occur as a complication of surgical treatment, such as a bypass, inadequate or impaired lung function, conduction disorders, structural heart disease, or inflammatory processes in the myocardium. Although factors such as early revascularization, the administration of inotropic drugs and mechanical support can improve the physiological condition of the patient, the mortality rate in the event of cardiogenic shock remains above fifty percent.

To stabilize the patient's condition, circulatory support systems have been developed that provide mechanical support and can be quickly connected to the circulatory system. They can improve the blood flow and perfusion of the organs, including the coronary arteries of the heart, and avoid a hypoxic state. For example, a blood pump may be connected to a venous access by means of a venous cannula and an arterial access by means of an arterial cannula for sucking or pumping the blood, respectively, to provide a flow of blood from a side with a low pressure, for example via an oxygenator, to a side with a higher pressure and thus support the patient's circulation.

However, the complexity and dynamics of the patient's own heart action require precise timing or coordination of the extracorporeal support. For example, blood flow through the heart's own coronary arteries, which normally supply the heart muscle with sufficient oxygen, generally occurs in the diastole of the cardiac cycle. A corresponding emptying of the left ventricle is therefore necessary. This is because if the filling pressure at the end of systole, or at the beginning of diastole, in the left ventricle is as low as possible, the coronary arteries can unfold their lumen as far as possible to increase the blood flow rate and oxygen supply. Accordingly, extracorporeal circulatory support for perfusion of the coronary arteries should be controlled in such a way that perfusion is preferably performed at the beginning of the diastole, wherein perfusion during systole should be avoided.

To control the extracorporeal support, measurement signals from an electrocardiogram (ECG) can be detected and used to determine characteristic amplitudes corresponding to different phases of the cardiac cycle. For example, an R-wave or R-wave characteristic of the systolic phase of the cardiac cycle is usually easily distinguishable from other phases of the cardiac cycle, for example in a QRS complex. The R-wave can thus be used, with a predefined latency, to control a blood pump in a successive diastolic phase.

However, the provision of an ECG signal can be impaired by various factors. For example, artifacts can form due to external influences, such that the corresponding amplitudes cannot be determined from the ECG signal. Such signal artifacts can occur, for example, as a result of a stimulation pulse from a pacemaker, which causes a signal level that conceals the patient's own ECG signal. Furthermore, amplitudes can also be determined from these artifacts or external signals with the corresponding signal level. In both cases a synchronization cannot be performed with sufficient safety for the patient, since either an artifact causes a latency that does not correspond to the cardiac cycle or the determination of a patient's own amplitude fails due to the artifact.

Thus, a control of extracorporeal circulatory support, which uses the amplitude as a trigger signal, may potentially be triggered at the wrong time, such that support is not provided in the intended or required cardiac cycle phase. To avoid this, the ECG measurement is reset accordingly when very high signals are detected or suspended for several cardiac cycles. However, this has the consequence that, at least for a certain period of time, there is no synchronization and thus no control of the extracorporeal circulatory support.

Accordingly, there is a need for early detection of possible interfering factors or interfering signals and to enable a qualitative ECG signal required for the control of the extracorporeal circulatory support even when artifacts occur.

### SUMMARY OF THE INVENTION

Based on the known state of the art, it is an object of the present invention to enable an improved stability of an ECG signal for an extracorporeal circulatory support.

This object is solved by the independent claims. Preferred embodiments are defined by the dependent claims, the description, and the Figures.

Accordingly, a control unit for extracorporeal circulatory support is taught, which is configured to receive a measurement of an ECG signal of a supported patient over a predetermined period of time and to provide said measurement for the extracorporeal circulatory support, wherein the ECG signal comprises a signal level from at least one ECG lead for each time point within a cardiac cycle. The control unit further comprises an evaluation unit which is configured to determine a signal difference of a signal level of a current time point and a signal level of the preceding time point and to compare the signal difference with a predetermined threshold value. The control unit is configured to provide the ECG signal with a predetermined signal level for the current time point and a predetermined number of subsequent time points, when the threshold value is exceeded.

Different cardiac cycles or cardiac actions can be recorded in the predetermined time period, wherein each time point is an absolute time or can also define relative time points, for example for the predetermined time period or a respective cardiac cycle from the beginning of a cardiac cycle to the end of a cardiac cycle. Each time point represents a measurement time point or detection point, wherein the measurement is preferably continuous in order to provide an improved temporal resolution for the extracorporeal circulatory support.

The measurement of the ECG signal can, for example, be received via an interface or by appropriate configuration of the control unit. For example, the control unit (i.e. control and regulating unit) can be directly communicatively coupled with at least one ECG lead or with an ECG device to receive detected ECG signals. The control unit is however preferably configured as part of an ECG device or in such a way that the ECG device can be attached to the control unit. Thus, the control unit can be used independently of the presence of other components and may adopt a compact design. Preferably, the ECG device is integrated in a single housing of a system for extracorporeal circulatory support, for example, in the sensor box in the form of an ECG card or an ECG module. Alternatively, however, the control unit can also be configured to receive an external ECG signal from the supported patient, for example from a cardiac monitor arranged outside of an extracorporeal circulatory support system. This allows the system to be configured even more compact.

The ECG measurement signals have a detected signal level and form corresponding data points, which can be processed and evaluated, respectively, by the evaluation unit. The evaluation unit can, for example, be formed as an integrated computation module and may include logic to evaluate the received signals and determine the signal difference. The signals can be recorded by the evaluation unit at least for a certain time period or also for the complete predefined time period or longer, for example, by means of a coupled or integrated storage medium or in a volatile working memory.

Furthermore, at least one preset threshold value is stored in the evaluation unit or in the control unit. As will be described below, different threshold values may be provided, for example, depending on the physiological condition, clinical picture or therapy of the respective patient or according to external influences. The signal difference can be an indicator for a relative slope or can be calculated as a derivative, wherein the signal level of the current time point is compared with the signal level of the time point immediately before or the ultimate time point, respectively. The determined or calculated difference is then compared with the stored threshold value to detect a potential artifact.

If the threshold value is accordingly exceeded, the presence of an artifact or an influence factor interfering with the ECG signal is assumed and the corresponding signal level of the ECG signal is overwritten with a predetermined signal level for a predetermined number of subsequent time points. Thus, a modified or corrected ECG signal is provided. The ECG signal can be transferred or transmitted to a device coupled with the control unit, e.g. via an interface, such that the corrected ECG signal can be analyzed and evaluated.

By determining or calculating the signal difference between the current time point and preceding time point, a potential interference is detected at the very beginning and a corresponding correction is carried out immediately, such that an ECG signal is provided which has a high validity and stability for an extracorporeal circulatory support and can be used for controlling a corresponding device. Thus, interfering signals from the ECG signal which do not correspond to the typical electrophysiological ECG morphologies can be faded out or blanked out, for example, to avoid incorrect R-triggering at this time point. For example, unipolar stimulation pulses can thus be blanked during bipolar (right ventricular) stimulation. The predetermined signal level typically corresponds to a normal value, which can be determined for a particular phase of the cardiac cycle, for example, based on stored empirical values. In this way, a reset of an ECG device of the extracorporeal circulatory support by exceeding an absolute value can be avoided, so that a stability of a control can be ensured.

Preferably, the ECG signal for each time point comprises a signal level from at least two ECG leads, wherein the evaluation unit is configured to determine the signal difference based on the sum values of the ECG leads.

Accordingly, the ECG signal may comprise at least a first measurement signal from a first ECG lead and a second measurement signal from a second ECG lead, wherein the first and second ECG leads are preferably spatially separated from each other. In other words, there are at least two data points for each time point within the predetermined time period. However, depending on the number of ECG leads being available, multiple data points can also be provided for each time point. For example, the predetermined time period can be defined by a treatment duration or a predefined number of detected cardiac cycles.

By adding or using the sum signal as part of a (spatial) signal averaging technique, a correction of single minor interfering signals is enabled, such that during the determining of the signal difference, certain fluctuations, which are for example anatomically and/or physiologically caused, can be taken into account and the accuracy of the ECG signal can be further improved. Thus, the ratio of the useful signal to the interfering signal can be improved by a factor of the square root of n for a number of n ECG leads, such that at least one amplitude change can be clearly or unambiguously determined, even for weaker measurement signals or fluctuations. With two ECG leads an improvement of \sqrt{(n=2)}˜1,41 can be achieved.

This improvement can be achieved, for example, in the presence of ideal noise with all frequencies, but can be reduced in the case of non-ideal noise signals, which can occur, for example, in the case of biosignal interference.

In other words, spatial or anatomical spacing can already ensure that the distance to certain interfering signals, for example external stimulation pulses of the heart, is improved and these interfering signals can thus be avoided as far as possible. These interfering signals can therefore not impair the determination of the signal difference. Furthermore, for the same (relative or absolute) time point of a single cardiac cycle, multiple ECG leads can be present to provide the ECG signal, such that for each time point a corresponding number of signal levels can be selected from particular ECG recordings or leads and used for processing.

The number of predetermined subsequent time points is preferably between 2 and 10 or between 2 and 20 time points, particularly preferably between three and five time points or corresponds to four time points. For example, at a sampling rate of 500 Hz, where each data point is 2 ms apart, four time points can correspond to 8 ms and ten time points to 20 ms.

Alternatively, however, the subsequent time period may be between 30 ms and 40 ms, which can be advantageous, for example, in the case of cardiac contractility modulation of the stimulation in the QRS complex with different polarities. Likewise, the number of predetermined subsequent time points can be selected in dependence of the sampling rate to achieve a predefined subsequent time duration. For example, the sampling rate can be 500 Hz or 1000 Hz.

The number of the subsequent time points preferably corresponds to a time, which blanks or suppresses an amplitude or a signal level of an artifact, such that the ECG signal to be provided does not contain significant interfering signals and these can be filtered out accordingly and a “blanking” is provided. Furthermore, the number of subsequent time points is preferably selected in such a way that particular amplitudes of an ECG signal or a particular cardiac cycle phase, respectively, are still present in the ECG signal and are not overwritten with the predetermined signal level. For example, it can thus be ensured that a QRS complex of an ECG signal and especially an R-peak or R-wave, which can be used as trigger signal for a control of an extracorporeal circulatory support, is present in the provided ECG signal. A number of predetermined subsequent time points may advantageously be chosen in a range of three to five time points and, in particular, four time points (or 6 ms to 10 ms or, in particular, 8 ms).

The number of predetermined time points can be determined, for example, on the basis of stored empirical values or a recorded and evaluated course of several cardiac cycles and, preferably, in a situation with different artifacts. Likewise, the number of predetermined time points can be set or adjusted dynamically in a variable manner and based on a recorded course, preferably also manually.

In order to further increase the stability of the ECG signal, the predetermined signal level can furthermore be the signal level of the preceding time point. In this way a native signal level is used, which corresponds to the cardiac activity or cardiac cycle phase of the respective patient and which also achieves in case of a successive determination of the signal difference after the subsequent time points, that a repeated exceeding of the threshold value at an expected and interference-free signal level due to the predetermined signal level is avoided as far as possible. That is in particular advantageous, whenever the predetermined number of subsequent time points amounts from three to five time points or, more specifically, corresponds to four time points. Thereby, an interference signal can be effectively blanked or suppressed, while a native signal level-due to the narrow time window of the blanked signal levels-can still be in the range of the signal level of the preceding time point.

As described in the above, various factors can influence the measured signal level. These factors can, for example, be either based on anatomical or physiological circumstances or can occur due to external influences. Preferably, the threshold value is hence characteristic for a slope of a particular interference signal.

It has been shown that the slope enables a distinction to be made between the native ECG signal of the heart activity and an ECG signal containing an artifact. In other words, the slope allows the earliest possible detection of the presence of a potential interfering signal, especially since it differs from the physiological signal. Accordingly, the corresponding signal level can be immediately blanked out and can be overwritten with a predefined signal level; thus, the stability of the provided ECG signal can be further improved and a reset of the control unit can be avoided.

The interfering signal can, for example, be a stimulation pulse from an external cardiac pacemaker, an implanted cardiac pacemaker, an implanted cardioverter, an implanted defibrillator, or a cardiac resynchronization therapy. In this way, despite a lack of coupling with the extracorporeal circulatory support or with an ECG device, an ECG signal with physiologically relevant signal levels can still be provided and complex processing and separation of the ECG signal is not required. If the slope or signal difference indicates that a stimulation pulse is being output, the stimulation-caused interference can be blanked by the corresponding number of predetermined subsequent time points.

The stimulation pulse can further be a unipolar stimulation pulse or a bipolar stimulation pulse. Combi-polar stimulation or multiphase stimulation pulses can also be provided, for example in the case of cardiac contractility modulation.

However, the slope can also be characteristic for pathophysiologically induced interferences or individual, spontaneous abnormalities. For example, the signal difference may exceed the threshold value due to certain movements or gravitational forces or even spontaneous, high P-waves or T-waves. Fusion stimulations, pseudo-stimulations or real stimulations can also lead to a corresponding exceeding of the threshold value. The threshold value can therefore be selected to include such interferences, alternatively or additionally. Preferably, the threshold value depends on the respective cardiac cycle phase or is variable within the cardiac cycle, wherein, for example, stored empirical values and/or recorded and evaluated ECG signals of the respective patient define the respective threshold value.

To evaluate the received ECG signal and to determine the signal difference, the evaluation unit can also be configured to determine the signal difference taking into account or factoring a recorded course of the signal levels and by using polynomial extrapolation. For example, not only the signal level of the ultimate or immediately preceding time point, but also the signal levels for earlier, preceding time points can be recorded and a trend line can be formed by means of a polynomial function, wherein a slope for the current time point is determined by means of the trend line. In this way, for example, stored empirical values can predefine the respective polynomial function and a determined signal difference exceeding the threshold value can be confirmed by means of the trend line, such that the validity of the determined artifact is further improved.

The at least one ECG lead preferably comprises a transthoracic ECG lead. If two ECG leads are provided and a corresponding sum signal is used to determine the signal difference, as described above, both ECG leads are preferably a transthoracic ECG lead. In this way, the detection of potential interfering signals is further improved, both by spatial separation and by proximity to a stimulation pulse in a potentially present cardiac pacemaker.

However, transesophageal ECG leads may also be provided. The number of ECG leads is not limited to the number of received or evaluated signal levels, such that in principle there is a choice of ECG leads to evaluate the ECG signal. For example, a plurality of transthoracic ECG leads (I, II, III, aVR, aVL, aVF, V1, V2, V3, V4, V5, V6) and (bipolar) transesophageal ECG leads (Oeso 12, Oeso 34, Oeso 56, Oeso 78) may be provided for electrographic analysis, wherein one or two of the respective ECG lead types can be used for the ECG signal or signal level, respectively.

In order to enable a high resolution of the signal level and a low latency of the ECG signal provision, the time interval between the time points preferably corresponds to a sampling frequency. For example, the sampling of the ECG signal and the corresponding signal levels can be performed at a frequency of 500 Hz as described above, such that there is a time interval of 2 ms between two respective time points per second. It has been found that the signal difference over such a short period of time already allows the detection of artifacts and a differentiation from physiological signals, so that an immediate correction of the received ECG signal or an interference signal, respectively, can be faded out or blanked immediately. Alternatively, the ECG signal can be sampled at a frequency of 1000 Hz to further improve the R-trigger stability. An interfering signal can be faded out or blanked for 4 to 20 ms by the predetermined signal level for a predetermined number of subsequent time points, which for example lies between 2 and 20, wherein blanking of four time points or 8 ms has proven to be particularly advantageous. During this time, however, a useful signal is provided by the predetermined signal level, such that subsequent signal levels can still be evaluated in case of an effective suppression of interference signals and, for example, a QRS complex can be detected from the ECG signal.

The control unit can furthermore be configured to provide the ECG signal in real time by evaluating the signal level of the current time point with respect to the ultimate time point. This not only ensures continuous provision, but may also prevent potentially dangerous interference signals for patients without significant delays. Furthermore, the temporal resolution can be further improved by an appropriate sampling frequency.

In order to enable processing or evaluation of the provided ECG signal, the control unit can also be configured to transmit the ECG signal to a detection unit for detecting a particular amplitude change of the ECG signal.

For example, the detection unit can be communicatively coupled with the control unit via an interface or be integrated into the control unit. Preferably, the control unit includes the detection unit.

In this manner, the provided ECG signal may be transmitted to the detection unit to detect a characteristic amplitude change within a cardiac cycle, wherein the amplitude change can be used as a trigger signal for the extracorporeal circulatory support.

Preferably, the detection unit is configured to determine a QRS complex as an amplitude change on the basis of the ECG signal. The number of the predetermined subsequent time points is thus selected in such a way that the QRS complex is not exceeded, e.g. not overwritten, or at least not completely, such that in particular an R-peak or R-wave can be detected.

Accordingly, from the QRS complex and in particular from a specific section of the QRS complex, an amplitude change characteristic of a respective cardiac cycle phase can be determined, which can be used to control the extracorporeal circulatory support, for example, by means of a control signal and a corresponding latency. Thus, the control can be provided at a specific moment in time and in a physiological state to provide maximum support for the cardiac performance.

Accordingly, an amplitude change should be determined, which can be used as a temporally stable trigger signal, for example, an amplitude change that is characteristic for a P-wave or R-wave. However, other amplitude changes may also be determined, for example, over a predetermined section of the ECG signal or from a prominent or distinctive point of the ECG signal. Preferably, however, at least one R-peak or R-wave is determined from the ECG signal, by means of which a trigger signal with a predefined latency time is output. The control unit may e.g. output a control or regulation signal for an operating parameter of a blood pump at a predefined time point after the detection of the R-peak, for example, the detection of the maximum amplitude, and the blood pump may be set or adjusted accordingly, typically with a delay.

The determination of the signal difference, preferably in real time, and the detection of a potential interference signal thus enables the ECG signal to correspond to physiologically relevant signal levels and that amplitude changes relevant for the control of extracorporeal circulatory support may be detected with high stability, such that a temporally stable, electrocardiogramally triggered and hemodynamically optimized synchronized extracorporeal circulatory support can be provided. For example, the amplitude change or the respective region in the (native) electrical excitation line can be characteristic of the systolic or diastolic phase of the heart, such that, for example, a blood pump may be actuated at a predetermined time point and in a predetermined phase and no overlap with other phases is caused. An erroneous detection of an amplitude change due to an interfering signal, which would cause the blood pump to be actuated in an unintended cardiac cycle phase, is effectively prevented by blanking of the corresponding signal levels.

It may also be provided that the received ECG signal is graphically displayed on a coupled monitor or display, for example by outputting a corresponding signal from the control unit. If the threshold value is exceeded, it may be specified or indicated for the respective time points that the signal levels are blanked or overwritten, such that a user can monitor the actual signal levels. This can be advantageous, for example, to monitor possible physiological influences or the timing of stimulation pulses. Furthermore, detected amplitude changes may be marked or identified in the respective cardiac cycles on the display. Accordingly, it is not only possible to determine whether the amplitude change occurred at the same or a similar time point in a respective cardiac cycle, but also whether it was determined at the correct time point, for example, at a maximum value and not at the beginning or end of an amplitude. Accordingly, the temporal stability can also be easily monitored visually using the markings.

Furthermore, the evaluation unit may be configured to determine the signal difference only within a predetermined time interval of a respective cardiac cycle.

The measurement of an ECG signal may be received in a continuous mode, whereby the signal difference is only determined in a section or a predetermined time interval of the cardiac cycle. Potentially occurring interference signals may be considered specifically for a particularly relevant cardiac cycle phase (e.g. during the time period of the QRS complex). For example, the predetermined time interval of at least one cardiac cycle phase of the ECG signal can be defined so that an amplitude change expected in this time interval, for example a QRS complex, can be detected. This allows to limit the evaluation of signal levels to the particular time interval, which not only facilitates data processing and accelerates processing, so as to ensure that the ECG signal is provided in real time, for example. It also enables higher accuracy in determining a potential interfering signal. For example, amplitude changes that are irrelevant for control purposes can be ignored or omitted, and a computing capacity can be used for particular signal levels or one or more time point(s) and corresponding cardiac cycle phases while providing a high resolution of the ECG signal. In an advantageous further embodiment, the time interval can be automatically preset by the evaluation unit on the basis of the heart rate and the signal levels.

The time interval may be optionally adjustable, for example, to extend or limit a fixed time period or time interval. Preferably, the control unit, in coupled state with a display, is thus configured to output to the display a signal representing successive cardiac cycles detected from the ECG signal for relative time points and an adjustable time range indication, which identifies the range of the evaluated signal levels. The evaluation unit is further configured to receive an adjustment signal from or via the coupled display and to determine the signal difference in the adjusted relative time range, when adjusting the time range for successive cardiac cycles. By adjusting the time window, for example, by shifting the limits on a horizontal axis, the time interval can be shifted and/or prolonged or shortened as required by the displayed cardiac cycles with respect to a relevant cardiac cycle phase. This provides the user with a certain flexibility and intuitive operation or usability to optimize the at least one amplitude change.

As a further safety measure, the control unit may be configured to provide the ECG signal for the current time point and the predetermined number of subsequent time points with the predetermined signal level, when an absolute threshold of the current signal level is exceeded. In other words, in addition to a detected or calculated signal difference, which may be e.g. characteristic for an interference signal, an absolute value may be provided, which indicates a potential system error when exceeded.

The above-mentioned object is furthermore solved by an ECG device with a control unit according to the invention as described above, wherein the control unit or the ECG device comprises a detection unit for detecting a particular amplitude change of an ECG signal, preferably for detecting a QRS complex, and wherein the control unit is configured to transmit the ECG signal to the detection unit.

The control unit may hence be formed as part of an ECG device or may be integrated into it and thus be coupled as an independent unit with the extracorporeal circulatory support or a corresponding system, for example via an interface. The ECG device can be formed or adapted as an ECG card or as an ECG module and can, for example, be communicatively coupled with and/or integrated into a sensor box of a circulatory support system.

According to the invention, a system for extracorporeal circulatory support of a patient is disclosed, wherein the system comprises a device for extracorporeal circulatory support, comprising a blood pump, which can be fluidically connected to a venous patient access and an arterial patient access and is adapted to provide a blood flow from the venous patient access to the arterial patient access, and an ECG device according to the invention. The control unit is communicatively coupled to the device and is configured to output a control signal for adjusting or setting the blood pump at a predetermined time point after the at least one amplitude change.

The control unit can also be accommodated in a console, which has a user interface for entering and reading out system settings, in particular, parameters of the blood pump and/or the ECG device. For example, the console may include a touch screen and/or a display with a keyboard that can be operated by a user. The control unit operates, actuates, controls, regulates and monitors the blood pump and enables the blood pump to be synchronized with the cardiac cycle of the respective patient.

For example, the control unit can record the received ECG signal and the heart rate, wherein the display shows the current ECG signal graphically and the current or averaged trigger frequency and/or trigger stability numerically. Furthermore, characteristic features of the ECG signal or the respective cardiac cycle can be emphasized or marked in the graphic display, so that, for example, a trigger signal determined as an amplitude change in a QRS signal can be marked in the form of an R-peak in the ECG signal or in the current cardiac cycle, respectively. Furthermore, further settings, such as the time interval between multiple amplitude changes or trigger signals, or the heart rate can be represented in the ECG signal, so that a user can monitor the control and regulation of the blood pump with regard to the physiological condition of the patient. In particular, exceeding the threshold value with regard to a particular signal difference as well as the corresponding output of a predetermined signal level for a predetermined number of subsequent time points can be represented.

The ECG device and the control unit can be formed as a sensor box, which can be connected via connectors to various sensors, such as pressure sensors of an extracorporeal circulatory support device, and an ECG device.

Furthermore, the output of the control signal or regulation signal for the extracorporeal circulatory support may cause an immediate setting or adjustment of a corresponding parameter or operating parameter of a coupled extracorporeal circulatory support device. For example, one or more pump drives or pump heads for blood pumps, e.g. non-occlusive blood pumps, in an extracorporeal circulatory support system can be controlled or regulated in this way. Thus, the ECG signal may be used to provide a desired blood flow rate to a corresponding cardiac cycle phase.

The blood pump can be connected to a venous access via a venous cannula and to an arterial access via an arterial cannula for sucking or pumping the blood to provide a blood flow from a side with a low pressure to a side with a higher pressure. Preferably, the blood pump is formed as a disposable or single-use item and preferably fluidically separated from the respective pump drive and easily couplable, for example via magnetic coupling. The control unit actuates the motor of the pump drive by outputting the corresponding signal and can thus cause a change in the speed of the blood pump.

The above-mentioned object is further solved by a method for providing an ECG signal for an extracorporeal circulatory support. The method comprises the following steps:
receiving a measurement of an ECG signal of a supported patient over a predetermined period of time, wherein the ECG signal comprises a signal level from at least one ECG lead for each time point within a cardiac cycle;
determining a signal difference between a signal level of a current time point and a signal level of the preceding time point;
comparing the signal difference to a predetermined threshold value; and
providing the ECG signal for an extracorporeal circulatory support,
   wherein the ECG signal is provided with a predetermined signal level for the current time point and a predetermined number of subsequent time points, when the threshold value is exceeded.

By calculating or determining the signal difference between the current time point and the preceding time point, for example, by means of an evaluation unit, a potential interference is detected at the very beginning and a corresponding correction is immediately carried out, such that an ECG signal is provided, for example, by a control unit, which has a high validity and stability for extracorporeal circulatory support and can be used for controlling a corresponding device. The predetermined signal level corresponds to a normal value, which can be determined, for example, on the basis of stored empirical values for a particular phase of the cardiac cycle. In this way, a resetting of an ECG device of an extracorporeal circulatory support upon exceeding an absolute value can be avoided, such that stability of a control can be ensured.

Preferably, the ECG signal comprises a signal level from at least two ECG leads for each time point, wherein the signal difference is determined by the sum values of the ECG leads.

Accordingly, the ECG signal can comprise at least a first measurement signal from a first ECG lead and a second measurement signal from a second ECG lead, wherein the first and second ECG leads are preferably spatially separated from each other. In other words, there are at least two data points for each time point within the predefined time period. However, depending on the number of ECG leads available, multiple data points may be provided for each time point. For example, the predefined time period can be defined by a treatment duration or a predetermined number of detected cardiac cycles.

By adding or using the sum signal as part of a (spatial) signal averaging technique, a correction of individual minor interfering signals is enabled, such that when determining the signal difference, certain fluctuations that are, for example, anatomically and/or physiologically caused, can be taken into account and the accuracy of the ECG signal can be further improved.

Thus, the spatial or anatomical spacing already ensures that the distance to certain interfering signals, e.g. from external stimulation pulses of the heart, is improved and these interfering signals can be avoided as far as possible, so that they do not impair the determination of the signal difference. Preferably, the ECG signal comprises a measurement signal of a transthoracic ECG recording or lead, so that the detection of a potential interfering signal, for example, a stimulation pulse, can be further improved.

The number of predetermined subsequent time points is preferably between 2 and 20 or 2 and 10 time points, and particularly preferably between three and five time points or corresponds to four time points. The number of subsequent time points preferably corresponds to a time period which suppresses or blanks an amplitude or a signal level of an artifact, such that the ECG signal to be provided does not contain any significant interfering signals and these are filtered out accordingly, such that a blanking is hence provided. Furthermore, the number of subsequent time points is preferably chosen or selected in such a way that particular amplitudes of an ECG signal or a particular cardiac cycle phase are still present in the ECG signal and are not overwritten with the predetermined signal level.

Furthermore, the predetermined signal level can be the signal level of the preceding or previous time point. In this way, the stability of the ECG signal can be further increased, since a native signal level is used which corresponds to the cardiac activity or cardiac cycle phase of the respective patient, respectively, and which also has the effect that, if the signal difference is determined successively after the subsequent time points, a renewed or repeated exceeding of the threshold value at an expected and interference free signal level is largely avoided due to the predetermined signal level.

The time interval between the time points may correspond to a sampling frequency and/or the ECG signal may be provided in real time. Thus, a continuous determination of the signal difference can be carried out at a high temporal resolution, which further increases the accuracy of the determined signal difference and keeps a possible latency as low as possible.

Preferably, at least one particular amplitude change is determined from the provided ECG signal, preferably a QRS complex, a P-wave and/or an R-wave.

By determining the signal difference and the potential overwriting of the signal levels for a predetermined number of time points, an ECG signal is provided which is as far as possible free of interfering signals and thus facilitates the detection of relevant, physiological amplitude changes. The corresponding at least one amplitude change can be used as a trigger signal for extracorporeal circulatory support, for example.

Furthermore, a control and/or regulation signal for a device for extracorporeal circulation support can be output at a predetermined time point after the at least one amplitude change. For example, a control signal for a blood pump can be output with a latency based on a determined or detected R-peak to enable the blood pump to be actuated in a corresponding cardiac cycle phase. The latency is preferably selected in such a way that the actuation takes place in a diastolic phase of the cardiac cycle.

Further advantages as well as possible variations and further embodiments of the methods have already been described in detail with regard to the control unit described in the above, so that a repeated description of the corresponding aspects is not necessary in order to avoid redundancies.

The above-mentioned object is further solved by a computer program product, which is stored on a computer-readable storage medium and contains instructions, which, when executed by a processor, effect the method steps according to the above-described method.

### BRIEF DESCRIPTION OF THE DRAWINGS

Preferred further embodiments of the invention are presented in more detail in the following description of the Figures, in which:

FIG. 1 shows a schematic representation of a control unit according to the invention;

FIGS. 2A and 2B show an electrocardiograma of two spatially separated ECG leads and a corresponding course of a signal difference; and

FIG. 3 is a schematic representation of a provision of an ECG signal when a threshold is exceeded.

### DETAILED DESCRIPTION OF PREFERRED EMBODIMENTS

In the following, preferred embodiments will be explained in more detail with reference to the accompanying Figures. In the Figures, corresponding, similar, or like elements are denoted by identical reference numerals and repeated description thereof may be omitted in order to avoid redundancies.

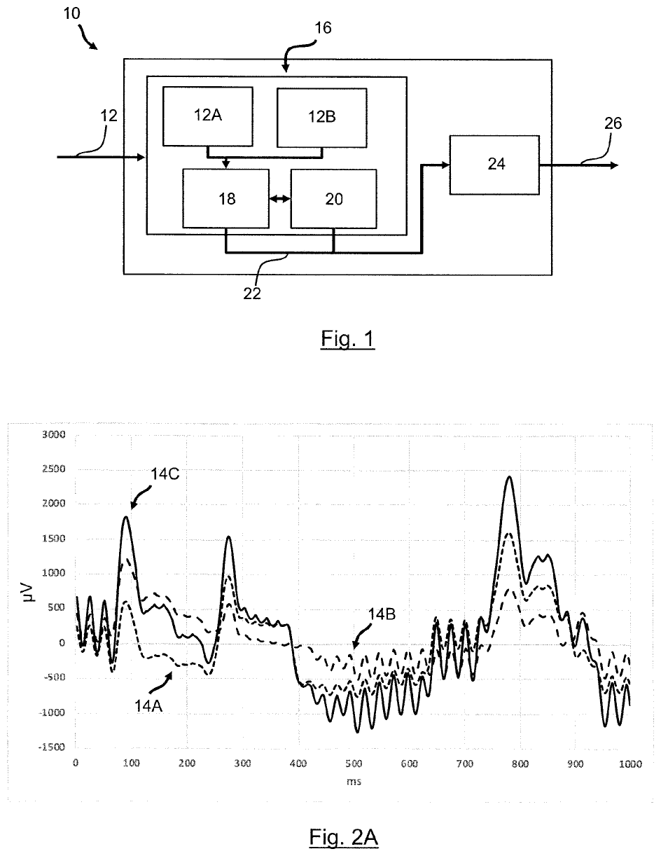

FIG. 1 shows a schematic representation of a control unit 10, which is configured to receive an ECG signal 12, as shown with the corresponding arrow. For example, the control unit 10 may include an interface for receiving measurement signals from one or more ECG leads or the control unit 10 may be formed as a corresponding ECG device. The control unit 10 according to the present example is formed as an ECG module, such that no special coupling is required for receiving the ECG signal 12. However, the ECG module can also include an interface (not shown), which enables a communicative coupling with an extracorporeal circulatory support system or an extracorporeal circulatory support device, such that this can be accordingly controlled or regulated by the control unit 10.

The received ECG signal 12 is read out by an evaluation unit 16 provided in the control unit 10, wherein the signal levels are continuously evaluated for each measured time point. The current signal level 12A is compared with the signal level of the previous or preceding time point 12B and a signal difference 18 is determined accordingly. The signal difference 18 can be used to optionally determine a slope, for example, if the time interval between the two time points is known. In this case the measuring points or time points correspond to the sampling frequency, which is 2 ms with 500 Hz for example.

The signal difference 18 is compared with at least one stored threshold value 20, which is provided for the evaluation unit 16. The threshold value 20 can, for example, be characteristic for a slope of a stimulation pulse of an implanted cardiac pacemaker and can be selected in such a way that it clearly differs from a physiological or native signal of the heart. For example, threshold value 20 can be in a range between 240 and 260 measured values, each measured value corresponding to a voltage between 2 and 3 μV. A threshold value 20 of about 250 measured values with a respective signal difference of about 2.8 μV has been found to be particularly advantageous, so that the threshold value 20 corresponds to a value of about 700 μV. If a signal difference 18 of, for example, 700 μV is determined, the threshold value 20 is exceeded in the present case.

If the threshold value 20 is exceeded, a predetermined signal level is provided in the ECG signal 12 for a predetermined number of subsequent time points, such that corresponding signal levels of an interfering signal are overwritten and omitted, i.e. a blanking is performed for these values. In the present case, the predetermined signal level corresponds to the signal level of the preceding time point 12B, such that a subsequent determination of the signal difference 18 is based on the patient's own and physiologically relevant signal levels and the threshold value 20 is not exceeded again. Thus, a correction of the ECG signal 12 can take place, so that the ECG signal 12 can also be used in case of interfering signals and a resetting of the control unit 10 for multiple cardiac cycles can be avoided. Accordingly, an ECG signal 22 is provided that is largely free of interference.

The provided ECG signal 22 is then transmitted to a detection unit 24, wherein the detection unit 24 is configured to determine an amplitude change from the provided ECG signal 22, for example, an R-peak or R-wave of a QRS complex. If the threshold value 20 is not exceeded, the provided ECG signal 22 essentially corresponds to the received ECG signal 12, but if the threshold value 20 is exceeded, the provided ECG signal 22 still contains physiological signal levels for a predetermined number of subsequent time points. The number of time points is selected in such a way that—according to the present example—in particular a QRS complex or at least an R-peak can be detected by the detection unit 24 and the subsequent time points do not overlap with the corresponding amplitude change.

The amplitude change that is determined or detected according to the present example serves as a trigger signal in order to provide a control signal 26 for an extracorporeal circulation support device with temporal stability. Accordingly, the control signal 26 can be output with a predefined latency, for example, to actuate a blood pump in a particular cardiac cycle phase. Thereby, for example, improved blood flow through the coronary arteries can be provided during a diastolic phase. For example, one or more amplitude changes can be determined, which are characteristic for an R-peak in the respective cardiac cycle, wherein the control signal 26 can be output accordingly as an R-trigger signal. The blanking of external signals or interference signals allows a control to be provided even if signal levels are present which would otherwise cause a reset and prevent the detection of an amplitude change.

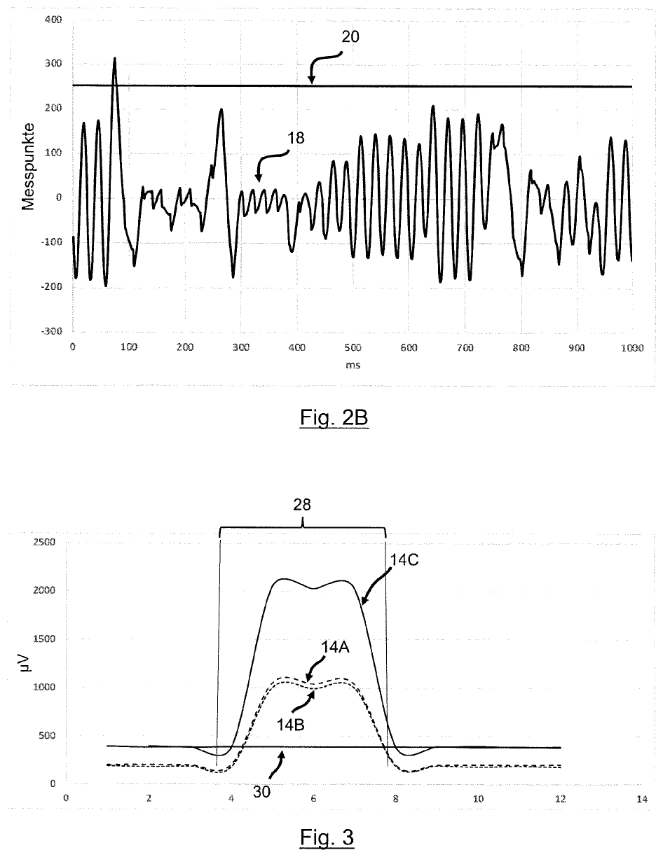

FIGS. 2A and 2B show exemplary electrocardiogramo spatially separated ECG leads 14A, 14B and a corresponding course of a signal difference 18. Although it is already possible to determine the signal difference 18 with an ECG signal from one ECG lead, the use of several ECG leads 14A, 14B offers a higher probability of detecting possible interfering signals, e.g. due to a stimulation pulse of a cardiac pacemaker.

By using the sum signal 14C to determine the signal difference 18, it is simultaneously ensured that small fluctuations, which may be physiologically caused, for example, do not lead to the threshold value being exceeded and that physiologically relevant signal levels can also be received and evaluated at any time. The sum signal 14C further improves the relationship or ratio between the useful signal and the possible interfering signal. The (corrected) sum signal 14C can accordingly be provided as ECG signal 22 for the subsequent determination of the signal difference 18 or also for a detection unit to determine an amplitude change from the (corrected) sum signal 14C. Nevertheless, the individual signal levels from the respective ECG leads 14A, 14B can still be transmitted to the detection unit or to a display for displaying the signal levels.

In the present case, the ECG leads 14A, 14B correspond to ECG leads II and III. Alternatively, or additionally, other ECG leads can be selected for receiving the ECG signal, e.g. transthoracic ECG leads I, aVR, aVL, aVF, V1, V2, V3, V4, V5, and V6 or bipolar transesophageal ECG leads Oeso 12 and Oeso 34. However, the number and type of leads is not to be regarded as limiting, such that in principle any selection of ECG leads can be used to determine the signal difference 18. This allows a spatially separated detection of measurement signals, both within one anatomical region and for different anatomical regions.

FIG. 2B shows the corresponding signal difference 18 graphically. As described above, the signal difference 18 is a difference in signal level between the current time point 12A and the preceding time point 12B. It can be seen that although fluctuations occur between the time points, but these are essentially physiological or measurement-caused fluctuations. At a time point of about 80 ms a signal difference 18 is determined, which exceeds the threshold value 20 and is accordingly detected as an interference signal. The provided ECG signal is corrected accordingly, as described above, for the corresponding time points.

FIG. 3 shows a further schematic representation of the provision of an ECG signal when a threshold value is exceeded. As shown in FIG. 2, in this example, signal levels from two ECG-leads 14A, 14B are also received and a sum signal 14C is detected accordingly. The X-axis shows the time points or measuring points, respectively, wherein the time interval between the respective time points corresponds, for example, to a sampling frequency and is about 2 ms. The signal levels are further processed by means of a polynomial function, so that the optional determination of a slope can be facilitated and/or determined with higher accuracy.

Although not shown, the determined signal difference at time point 4 exceeds the stored threshold value, such that a potential interference signal has been determined. Accordingly, the ECG signal for time point 4 and a predetermined number of subsequent time points 28 is overwritten with a predetermined signal level 30. In this case, the preset or predetermined signal level 30 is used for four subsequent time points 28, wherein the predetermined signal level 30 corresponds to the signal level of the preceding time point of the sum signal 14C.

In this way, the detected interference signal is blanked in the provided ECG signal, such that an improved stability of the provided ECG signal with physiologically relevant signal levels can be transmitted to a detection unit and thus trigger signals with high temporal stability can be provided. The improved temporal trigger stability based on the determination of a signal difference, which can take place continuously and in real time, can thus be particularly advantageous for the precise control of an extracorporeal circulatory support, wherein interfering signals can be blanked or corrected. For example, interfering signals resulting from intermittent stimulation, such as bipolar right ventricular stimulation, can be suppressed or corrected in a patient with an implanted pacemaker with heart failure and coronary artery disease, but with normal left ventricular pumping function.

Where applicable, all the individual features depicted in the exemplary embodiments may be combined and/or exchanged without leaving the scope of the invention.

### LIST OF REFERENCE NUMERALS

10 Control unit
12 Received ECG signal
12A Current signal level or current time point
12B Preceding signal level or preceding time point
14A Signal level of the first ECG lead
14B Signal level of the second ECG lead
14C Sum signal of the first and second ECG leads
16 Evaluation unit
18 Signal difference
20 Threshold value
22 Provided ECG signal
24 Detection unit
26 Control signal
28 Subsequent time points
30 Predefined signal level

1. A control unit (10) for an extracorporeal circulatory support, which is configured to
receive a measurement of an ECG signal (12) of a supported patient over a predetermined period of time and to provide said measurement for the extracorporeal circulatory support, wherein the ECG signal (12) comprises a signal level from at least one ECG lead (14A, 14B) for each time point within a cardiac cycle,
wherein the control unit (10) comprises an evaluation unit (16) which is configured to determine a signal difference (18) of a signal level of a current time point (12A) and a signal level of the preceding time point (12B) and to compare the signal difference (18) with a predetermined threshold value (20),
wherein the control unit (10) is configured to provide the ECG signal (22) for the current time point and a predetermined number of subsequent time points (28) with a predetermined signal level (30), when the threshold value (20) is exceeded.

2-26. (canceled).
